(19) United States
(12) Patent Application Publication
(10) Pub. No.: US 2025/0230392 A1
(43) Pub. Date: Jul. 17, 2025

(54) MICROFLUIDIC CHIP

(71) Applicant: PROBIONT OY, Helsinki (FI)

(72) Inventor: Benedek Poor, Helsinki (FI)

(21) Appl. No.: 18/703,740

(22) PCT Filed: Oct. 29, 2021
(86) PCT No.: PCT/FI2021/050735

§ 371 Date: Apr. 23, 2024

(51) Int. Cl. C12M 3/06; B01L 3/00; C12M 1/00; C12M 1/04; C12M 1/12; C12M 1/34; C12M 1/36

(52) U.S. Cl. CPC C12M 23/16; B01L 3/502707; C12M 23/06; C12M 23/22; C12M 23/24; C12M 23/34; C12M 41/36; C12M 41/48; B01L 2200/027; B01L 2300/0819

(57) ABSTRACT

The microfluidic device comprises a substrate and functional units. Each functional unit comprises first chambers arranged to be in fluid contact by a first channel and second chambers arranged to be in fluid contact by a second channel. A microchannel array is arranged to connect the first channel and the second channel. A method for culturing cells comprises providing first chambers with immune cells, providing second chambers with cancer cells, incubating the device and counting the immune cells and/or cancer cells while observing the migration of the immune cells to the cancer cells. In the manufacturing method, substrate is provided and casted to define functional units. Each functional unit is provided with first chambers arranged to be in fluid contact by a first channel, and second chambers are arranged in fluid contact by a second channel. A microchannel array is arranged to connect the first channel and the second channel.

### TECHNICAL FIELD

The application relates to a device for culturing cells. More specifically, the application relates to a device for culturing cells, spheroids, and organoids in vitro.

### BACKGROUND

Microfluidic chip has been applied as one solution for studying the efficacy of immunotherapy by evaluating immune cell migration towards cancer cells. One of the state-of-the-art chips was developed by Businaro et al. (Lab Chip 13 (2013) 229-239) and it has been used for testing the effects of several immunotherapeutic approaches (e.g. Lucarini et al. J. Investig. Dermatol. 137 (2017) 159-169). Matrigel has often been used in these assays to provide 3D environment for the cancer cells. Also fully human in vitro microfluidic chip assays have been used to test immunotherapy for personalized medicine purposes (Al Samadi et al. Experimental Cell Research 383 (2019) 111508). In general, microfluidic chip designs, with or without immunomodulators, have been used to test both the immune cell migration towards cancer cells and their cytotoxic activity. The chip design is important as on the one hand sample volumes must be large enough for accurate and repeatable measurements, but on the other hand the chip design should be small enough to fit onto a microscope slide for imaging and measurements. Present designs, such as Businaro et al. only enable the measurement of a single sample on a chip.

The prior art devices may therefore offer only limited possibilities for imaging, while loading of these devices may also be cumbersome. Traditionally, microfluidic chip devices are loaded by hand such that each chamber is separately loaded by pipettes, which may be time consuming, laborious, and/or subject to human error. In attempting to use automatic pipettes for device loading, a problem of formation of air bubbles is usually seen, leading to unreliable loading via automatic pipettes. Additionally, known devices have limited gas exchange and/or limited nutrient provision for the cells.

### SUMMARY

It is an object of the disclosed invention to provide an improved device and to alleviate at least some of the problems associated with cell culturing devices of the prior art.

According to an aspect of the application a microfluidic device comprises a gas permeable substrate with one or more functional units. Each functional unit comprises two first chambers that are arranged to be in fluid contact by a first channel. Each functional unit comprises two second chambers that are arranged to be in fluid contact by a second channel. Each functional unit comprises a microchannel array that is arranged to connect the first channel and the second channel. The microchannel array, the portion of the first channel in fluid connection with the microchannel array, and the portion of the second channel in fluid connection with the microchannel array form an operational part of each functional unit.

According to another aspect of the application, each functional unit of the microfluidic device may be a separated unit. This means that any one functional unit is not in fluid contact with any of the other functional units. The units can even be arranged to be loaded independently. The width of the microchannel array may be in the range of 30-9000 μm, preferably 3000-7000 μm, more preferably 4000-6000 μm. Alternatively, the width of the microchannel array may be in the range of 30-2000 μm, preferably 50-100 μm. The microchannel array contains microchannels whose width may be 10-15 μm, preferably 11-13 μm, height may be 5-20 μm, preferably 10-15 μm, length may be 10-2000 μm, preferably 10-1000 μm, and the spacing between each of the microchannels may be 20-40 μm,

According to another aspect of the application, each of the first chambers of the microfluidic device may have a height of 100 μm-3 cm, and an essentially circular cross section with a radius of 0.5-10 mm, preferably 3-5 mm or an area spanning over 0.7-314 mm², preferably 28-78.5 mm² of any other 2D shape. Each of the second chambers of the microfluidic device may have a height of 100 μm-3 cm, and a surface area corresponding to an essentially circular cross section with a radius of 200-400 μm, or a radius that corresponds to the outer diameter of the radius of the loading device that is used to load each of the second chambers. The first channel of the microfluidic device may have a height of 10 μm-300 μm, preferably 170 μm-190 μm, such as 189 μm, and a width of 200-3000 μm, preferably 1190 μm. The second channel of the microfluidic device may have a height of 10 μm-300 μm, preferably 170 μm-190 μm, such as 189 μm, and a width of 100-1000 μm, preferably 490 μm. The length of the first channel of the microfluidic device may be at least the length of the microchannel array, and the length of the second channel of the microfluidic device may be at least the length of the microchannel array.

According to another aspect of the application, at least a portion of the microfluidic device, optionally essentially all or most of the device apart from the first and second chambers, may be transparently covered.

According to an aspect of the application, the microfluidic device may be configured to be arranged on a slide for microscopy. According to another aspect of the application, the microfluidic device may be configured to be arranged on a microplate. Depending on the experiment, the microfluidic device may comprise 1-384 functional units.

According to an aspect of the application, the microfluidic device may comprise PDMS. PDMS or suitable derivative of PDMS may provide adequate gas permeability. This facilitates gas exchange which is crucial in cell culturing.

According to a cell culturing method, a microfluidic device is provided with at least one first chamber with at least immune cells, and at least one second chamber with at least cancer cells. The microfluidic device is incubated, whereafter the immune cells and/or cancer cells are counted while observing the migration of the immune cells to the cancer cells. At least immune cells for the at least one first chamber and/or at least cancer cells for the at least one second chamber are provided by one or more automatic pipettes.

According to a manufacturing method of a microfluidic chip substrate is provided. The thickness of the substrate is limited to 30 μm-3.5 mm. The substrate is worked to define one or more functional units. Each functional unit comprises two first chambers arranged to be in fluid contact by a first channel. Each functional unit comprises two second chambers arranged to be in fluid contact by a second channel. Each functional unit comprises a microchannel array arranged to connect the first channel and the second channel. Each functional unit comprises an operational part arranged to connect the portion of the first channel in fluid connection with the microchannel array and the portion of the second channel in fluid connection with the microchannel array (114). According to the method the substrate is then cured.

Automatic pipetting solutions of the prior art have long channels. Long channels cause more pressure drop while pipetting. The pressure drop along a shorter channel is less. When the pressure drop is smaller, it enables better control of the liquid flow while injecting the liquid Into the device. This is an important point as automatic pipettes are purposed to “drop” liquid with minimal resistance, not for injecting into cavities with back pressure. Therefore, more viscous liquids, which are typically used in cell culture experiments, can be challenging to inject into a microfluidic channel. For this reason shortening of the channels results in more reliable filling process and less chances of bubble formation while filling. Another point is that when less volume is needed from the sample, less drug is required for the tests. This is a clear advantage for lowering the cost of some in vitro tests, where the cost of the drug may be significant, e.g., immune checkpoint inhibitors or other antibody based therapeutics.

Another improvement in the operation of the new device is that the priming time of the microchannels, i.e. the disappearance of the air pockets initially present in the microchannels following the loading of the liquids from both sides, is quicker, and therefore enables faster operation of the device.

The main differences of the disclosed solution compared to prior art is the improved device architecture (referring to placement, size, shape, and/or dimensions of channels and/or chambers), specifically at least channel architecture, enabling the placement of a plurality of, e.g., up to 384 functional units on a single chip. The functional units comprise two first chambers connected by a first channel and two second chambers connected by a second channel. The first and the second channel are connected by a microchannel array. The term “connected” may herein generally refer to a fluid connection.

It was discovered by the inventor that the side chambers and side channels of the prior art included for the purpose of to be filled with fresh medium may be unnecessary for providing sufficient cell viability. Eliminating these channels may result in the elimination or reduction of the influence of the nutrient concentration gradient formed during operation of the device from the opposite direction where the cells are to be migrating to, driven by chemical signals originating from the other side of the microchannel array. The devices according to the invention may therefore be realized without such side channels and reduced nutrient concentration gradient.

Prior art assumes that large side containers/chambers are needed to provide sufficient nutrient supply for the cells. Therefore more functional units could not have been fitted on the slide. In the disclosed microfluidic device according to the present construct, due to the geometry of the chambers and/or channels, specifically height and/or volume, more liquid per amount of cells may be loaded into the chambers and into the channels, enabling provision of sufficient nutrients while retaining an overall compact size of the functional units. The liquid may be e.g. matrigel or other viscous media with added gel forming additives, such as fibrinogen. In prior art solutions, a volume of 150 μL has 20 been previously used. Here with the proposed construct the total volume can be 1-1000 μL, preferably 1-500 μL, more preferably 1-100 μL. The cell content of the cell suspension in the prior art is 3 μL for cancer cells and 150 μL for immune cells. In the present construct the cell content of the cell suspension volumes may be 0.1-149 μL, preferably 0.1-2.9 μL, more preferably 0.5-2 μL, even more preferably 1-2 μL.

The liquid may be a cell culture medium. The cell culture medium is chosen based on the cell or organoid types and assays. In general, all the cell culture media comprise a carbon source as a source of energy, amino acids as building blocks of proteins, and vitamins to promote cell survival and growth. In addition, a balanced salt solution of the medium should maintain an isotonic mixture of ions to optimize osmotic pressure within the cells and provide essential metal ions to act as cofactors for enzymatic reactions. Furthermore, a buffer (e.g. Bicarbonate or HEPES) is used to maintain a balanced pH in the media.

It is important that the liquid loaded is destined to form a hydrogel suitable for 3D cell cultures. These hydrogels may be, selected from a list containing, but not limited to nanocrystalline or nanofibrillated cellulose, chitosan, alginate, hyaluronan, polypeptide, collagen, gelatin, fibrinogen, agarose, or their functionalized versions based on any combinations or hybrid forms of these. Suitable synthetic hydrogels may be, for example, polyethylene glycol, polyurethane, poly(-ε-caprolactone) or any other functionalized versions or combination of these. Other components of the media may include for example: laminin, entactin, heparan sulfate proteoglycan, tumor growth factors.

Drugs, for example, pembrolizumab, nivolumab, cemiplimab, atezolizumab, avelumab, durvalumabor and/or any other monoconal antibody based drugs with the ability of blocking cancer or immune cell receptors with a direct or indirect therapeutic purpose may be added to the cell suspensions in order to evaluate drug efficacy.

It is essential that the hydrogel formation occurs post injection of the cell suspensions into the microfluidic channels. The gel formation can be initiated by heat or light. Cold (4-25° C.) cell suspension may be injected into the chamber followed by placing the device in 37° C. Heat initiates the gel formation and thus the hydrogel emulating the cancer microenvironment is formed.

Each of the functional units may be separated, i.e. not in physical contact with any other functional unit of a device. Thus, each of the functional units may be suitable to be loaded with samples of any type independently. This is an advantage over the prior art where two sides can communicate through a central channel, restricting the use for only one type of cell suspension loaded in the central channel. With the present invention, each functional unit may be loaded with same or similar sample types or each functional unit may be loaded with a different sample type, for instance.

Samples may here refer to suspensions comprising cells or other organelles capable of spontaneous self-migration or suspensions of chemical components added to cause any alteration of the observed physiological functions. The sample types may refer to samples different in their composition of the above, which therefore should be examined individually. With the present invention, the sample types may be placed in separate functional units but on the same device.

Typically, the type or the dosage of e.g. a drug added may vary sample by sample administered on the same device. Other scenarios may include samples with varying cell types or differences in cell types or concentration or type of other chemical factors defining the cell (or cancer) microenvironment.

In advantageous embodiments of the invention, the length of the operational part of the first and second channels (length of the section of the channels where they are interfaced with each other and connected through the microchannel array, i.e. the width of the microchannel array) may be shortened compared to the prior art, enabling faster imaging, capturing the site quicker, therefore reducing photobleaching and UV light exposure on the cells loaded. The new design also enables simultaneous imaging on one chip.

The unique architecture of the device, such as size and geometry of the microfluidic channels, enables the placing of 2-10, such as 3-5 or 6-10 functional units in a single microfluidic chip (depending on the embodiment), to e.g. fit the size of a standard microscope slide. The surface area of a tray holder of a microscope is or is compatible with 85.5×127.5 mm. So the microfluidic chip size range may be defined between a standard microscope slide 26×76 mm and the whole size of the tray which can either incorporate a larger slide with multiple functional units or several standard microscope slides placed on an adapter frame with the overall surface area of 85.5×127.5 mm. The sizes or architecture of the device and components, such as channels and functional units, could be tailored to the experiment and the microscope slide size, e.g. to fit standard microscope and/or microscope scanner device stages.

If functional units of the microfluidic device are placed on a microplate instead of microscopic slide, one may construct a microfluidic device with, e.g., 1-384 functional units. An additional benefit of such microplate approach is that in addition to pipetting, also imaging may be further automized, e.g., using robotic pipetting and a plate reader. The invention may therefore provide a novel way to construct a microfluidic chip device that may enable more than one sample testing and/or imaging on a single chip.

By providing a plurality of, such as more than one or two functional units, parallel and simultaneous monitoring of the immune response (i.e. observation of the migration of the immune cells to cancer cells) under e.g. 3 to 10 different conditions (different drug combinations or dosages) becomes possible. The size and number of the functional units may be determined by the area of the chip, microscope stage, and/or microscope scanning area. Depending on the type of the drug, it can be mixed with either or both the immune cells and the cancer cells containing medium prior to injecting. When using the device in other scenarios, a mixture or combination of several drugs may be added to the liquids before injecting to the chambers on the both sides.

With a plurality of functional units in one device, different samples can be monitored simultaneously without the need of removing e.g. a slide onto which the device is placed from the readout device (e.g. a fluorescence microscope or a microscope slide scanner) between monitoring of different samples.

The operational part is defined as the whole channel sections from both sides along the microchannel array, i.e. those parts of the first and second channels in fluid contact with the microchannel array. The length of the operational part of the channels in the device according to embodiments of the invention may also be beneficial for the imaging, as imaging a single unit takes less time requiring less shots to be taken and less images to splice together. Assuming the use of a wider objective for the readout device, one imaging shot may capture the whole operational part and no splicing of images will be needed. Alternatively, only specific section may be selected for imaging.

The presented chamber and channel architecture may enable the whole functional unit to be at least 30%, or preferably at least 50%, or more preferably at least 75% shorter than in the prior art.

Through shortening the channel lengths, there is a clear advantage in being able to reduce the amount of sample needed for to be injected into the device.

This is especially important, when the number of cells available in the sample (e.g. separated from a biopsy sample) is limited, but equal concentration of cells in the media has to be maintained for the proper functioning of the device. With the proposed construct it is possible to provide the circumstances in which the cell populations on the two sides of the microchannel array can be interfaced properly with each other. That is, the cells are evenly distributed on each side of the microchannel array. The width of the microchannel array, i.e. the length of the operational part of the first and second channels may be 30-9000 μm, preferably 3000-7000 μm, preferably 4000-6000 μm.

According to an embodiment the width of the microchannel array may also be 30-2000 μm, preferably 50-100 μm. This may be advantageous when sample volumes are very small, but a high concentration ratio between the cell volume and the total volume is required by the assay.

Further, with the provided device where channel geometries (first, second and/or microchannel array) provide smaller volume channels than in the prior art, the volumes or cavities require less fluid for filling the channels, therefore the device works with smaller samples as well, which is a clear benefit if limited number of cells, spheroids, or organoids are available or needed, or the availability or cost of the drug or other substance examined is limited.

Furthermore, the disclosed microfluidic device has channel geometry (specifically shorter channels or at least shorter operational parts of channels) which may enable reliable filling of the first and/or second chambers with automatic pipettes. The loading should be done while preventing bubble formation in the channel that can be hard to achieve if using automatic pipettes. The length of prior art channels are longer, and therefore the pressure drop of the injected fluid if higher viscosity is larger along the channel, and can exceed the capabilities of some automatic pipettes for creating a laminar flow while loading. This may result the generation of bubbles while loading the prior art devices. However, the present invention may enable laminar flow along the channels and essentially prevent any turbulent flow while injecting, and thus bubble formation may be essentially eliminated or at least reduced.

According to an embodiment the surface area of the chambers may vary depending on their intended load volumes. The chambers that have small volumes may have a small surface area or radius, typically corresponding to an essentially circular cross section with a radius of 200-400 μm, or matching the outer diameter of the radius of the loading device (e.g. a pipette), to provide sealing for the tip when the cell suspension is injected. The proper sealing is crucial because it prevents bubble formation when injecting. On the other hand, chambers with large intended volumes may have a larger surface area in order to facilitate gas exchange on the chamber surface.

The device may provide a platform for predicting the efficacy of drugs intended to be used for immunotherapeutic purposes in individual patients of cancer and other diseases. It is intended to be used as an in vitro or ex vivo microfluidic platform for developing personalized strategies in the treatment of diseases where the evaluation of the triggered immune response in the individual patients is essential for identifying the optimal drug type, dosage and combination prior to their administration to the individual patient.

The device, according to one embodiment of the invention, may comprise glass, PDMS, Flexdym polymer, polystyrene, polypropylene, polycarbonate, PMMA, ceramics, silicone, and/or thiol-ene. The material of the functional sections of the channel may be made out of PDMS or suitable derivative of PDMS providing adequate gas permeability. When considering a hybrid assembly of the device other materials: glass, flexdym, Thiol-enes or any derivatives of that, COC, or any block co-polymer with sufficiently low turbidity and high transparency, may be used.

The previously presented considerations concerning the various embodiments of the device may be flexibly applied to the embodiments of the method of manufacturing the device mutatis mutandis, and vice versa, as being appreciated by a skilled person.

### BRIEF DESCRIPTION OF THE DRAWINGS

The figures are presented to illustrate the disclosed embodiments, and are not to be taken to be limiting the scope of their use. The figures are not in any particular scale.

FIG. 1 shows at 1A a prior art device, while 1B is a tilted side view of an embodiment of the microfluidic chip device of the invention on a microscope slide.

FIG. 2 is an enlargement of a portion of the tilted side view of the embodiment of the microfluidic chip device in FIG. 1B.

FIG. 3 is an enlargement of a portion of the tilted side view of the embodiment of the microfluidic chip device in FIG. 2.

FIG. 4 shows enlargements of the tilted side view of a portion of one embodiment of the microfluidic chip device.

FIG. 5 is a top view of one embodiment of the microfluidic chip device on a microscope slide.

FIG. 6 is a flow chart of a method of culturing cells according to one embodiment of the invention,

FIG. 7 is a flow chart of a method of manufacturing a device according to one embodiment of the invention.

### DETAILED DESCRIPTION

The solution is described in the following in more detail with reference to some embodiments, which shall not be regarded as limiting.

In the description “chamber” and “circular well” may be used interchangeably. In the description “cell” may be used as an extended term including cells, cell aggregates, spheroids, and organoids. Within context of this description term “comprising” may be used as an open term, but it also comprises the closed term “consisting of”.

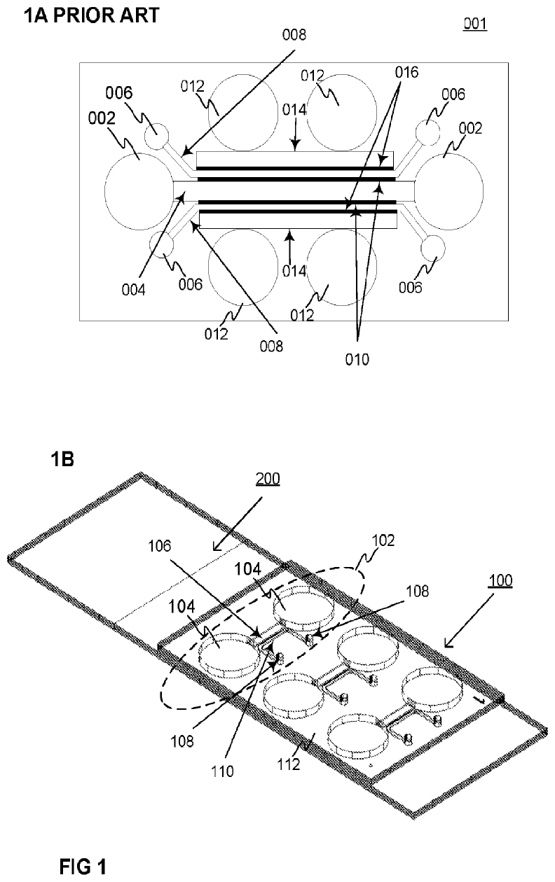

FIG. 1A shows a device 001 of the prior art. The device comprises at least two primary chambers 002 that are connected by a primary channel 004. The device also comprises two pairs of secondary chambers 006 that are connected by secondary channels 008. At both sides of the primary channel 004, the primary channel 004 is connected to the secondary channels 008 through primary microchannel arrays 010.

The device 001 also comprises a number of side containers or side chambers 012. The side chambers 012 are connected to the secondary channels 008 by side channels 014 and secondary microchannel arrays 016. The side chambers 012 are provided adjacent to the cell containing primary and secondary channels, the side chambers 012 communicating with the secondary channels 008 through the secondary microchannel arrays 016, and serve as a fresh source of nutrients and for the purpose of hydrating the gel matrix of the cell containing channel(s) or chamber(s), also aiming to prevent its shrinkage during use.

In the prior art device, the constituents 002-016 may be considered to form one functional unit, which is suitable for studying one cell type or one cell sample or cell interaction type. Due to the space taken up by the functional unit, the number of functional units that can be employed at the same time, e.g. on one microscope slide, is limited to only one.

FIG. 1B shows a device 100 according to one embodiment of the invention, where the device is placed on a microscopic slide 200. The device 100 comprises one or more functional units 102 each functional unit 102 comprising two first chambers 104 arranged to be in fluid contact by a first channel 106 and two second chambers 108 arranged to be in fluid contact by a second channel 110. A microchannel array (not clearly visible in FIG. 1) is arranged to connect the first channel and the second channel.

A functional unit 102 may consist of the two first chambers 104, first channel 106, two second chambers 108, second channel 110, and microchannel array.

A device 100 and functional unit 102 may be provided without any side chambers and side channels, such as those depicted in connection with FIG. 1A.

The first and/or second chambers 104, 108 may be essentially cylindrical in shape, having a circular base with a selected radius to provide a circular well for receiving a fluid. The cylinder wall may define the height of the chambers. The first and/or second chambers 104, 108 may also embody some different shape, e.g. the base may be rectangular. The size of e.g. the base shape together with the wall height may be selected to define the volume of each of the first and/or second chambers 104, 108.

The device 100 of Fig. comprises three functional units 102. Also some other number of functional units 102 may be employed. The size of the functional units 102 or the size of the channels and/or chambers may determine the number of functional units that may be employed for a certain use case scenario.

The device 100 may be considered to comprise a substrate 112 forming at least the base of the device 100. The substrate material may e.g. comprise silicone (polydimethylsiloxane (PDMS)) and the substrate material may define the structures arranged on the device, i.e. the chambers and channels. The thickness of the substrate may be 30 μm-3.5 mm. The thickness of the substrate may be variable, i.e. the substrate may be locally thinner. The floor thickness may be 0.03-3 mm, preferably 0.05-2 mm, more preferably 0.1-0.9 mm. The roof thickness may be 0.05-20 mm, preferably 0.1 to 3 mm, more preferably 0.11 to 2 mm. The thickness of the channel and/or chamber walls may be 10-50 μm.

In use, the first chambers 104 may be provided with an immune cell suspension and the second chambers may be provided with cancer cells. An immune cell suspension may comprise at least immune cells in a medium. The immune cell suspension may further comprise proteins and the mixture of other organic and inorganic compounds added for the purpose of emulating the in vivo microenvironment of the cancer cells.

Cancer cell suspension may comprise at least cancer cells suspended in a medium and hydrogel forming additives. For example, matrigel or other viscous media with added gel forming additives, such as fibrinogen, may be used.

The immune cells in the immune cell suspension prior to injecting may be stained with first cell tracker dye. The cancer cells in the cancer cell suspension may be stained with second cell tracker dye. When the immune cell interacts with the cancer cell and the outcome of the interaction is the death and consequent lysis of the cancer cell, the emitted color of the fluorescent dye used for staining the cancer cell will change. This may be monitored under fluorescence microscope, scanner or other suitable alternative readout device.

The device may also be used for other cell culturing purposes than cancer cells and immune cells. The samples employed may e.g. comprise materials emulating a studied physiological phenomenon. The samples may comprise drugs, hormones, antibodies, or cancer microenvironment specific proteins, for instance.

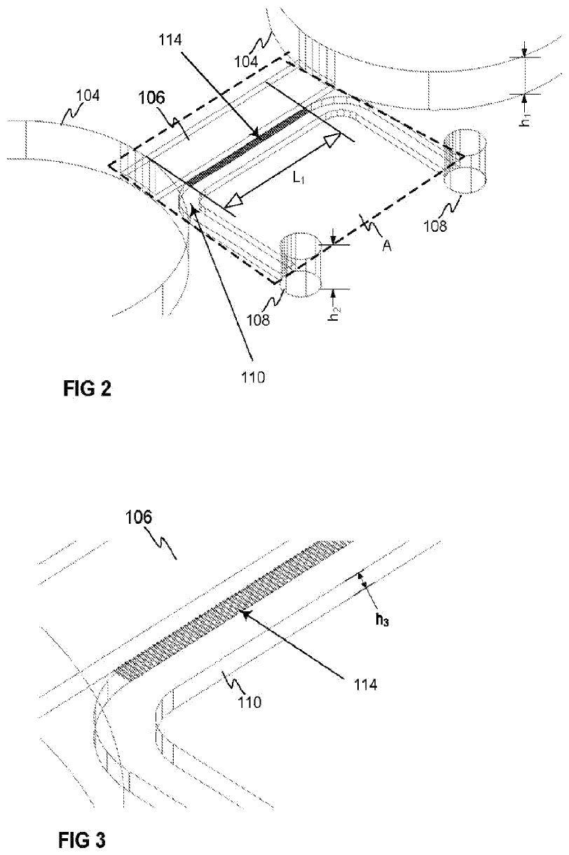

FIG. 2 shows a portion of an enlargement of the tilted side view of the embodiment of the microfluidic chip device 100 in FIG. 1. The first chambers 102 are depicted partially, along with the second chambers 108, first channel 106 and second channel 110. The microchannel array 114 connecting the first channel 106 and second channel 110 is also schematically depicted.

The overall thickness of the device 100 may be determined by the desired pipetting volumes, or volumes of fluid that are to be delivered to the chambers 104, 108. The pipetting volumes may be used to determine the heights and/or radii (or other dimensions in case of other shapes than cylindrical chambers) of the first and/or second chambers. The pipetting volume may be 1-1000 μL for each of the first and second chambers. A volume enclosed by first and/or second chambers may be selected to essentially correspond to a pipetting volume. The overall thickness of the microfluidic device may be 130 μm-2 cm. The overall height/thickness of the device 100 may be dependent on the height of the chambers 104, 108.

According to an embodiment the ranges defined for the channel lengths may represent the type of experiment in question. In other words, cells, spheroids, and organoids have different diameters, and therefore, the channel geometries may vary depending on the diameter of the injected cells, spheroids, or organoids. For those device versions where organoids or spheroids are injected, the channel depth and the channel width of the channel 110 may be the diameter of the spheroid times two. The sample volume for channel 110 may equal to the total volume and the volume of channel 106 may equal to the sample volume. The sample volume may be injected directly into the channel, without filling the reservoir 106. The total volume for this section may be the volume of channel 106 plus the two reservoirs, which may be filled with the cell culturing suspension without the cells.

The height of the chambers 104, 108, together with the radii, determine the amount of fluid (cell culture) sample that may be loaded depending on the length of the experiment. The height h₁ and/or h₂ of each of the first 104 and second 108 chambers, respectively, may be. The height of the chamber 104 can be between 100 μm and 3 cm (if a cylindrical ring is attached to the base). the height of the chamber 108 can be the same or different from the chamber 104 within the above range.

In one embodiment, at least a portion of the device 100 may be covered with an essentially transparent material such as glass or transparent polymer. Essentially all or most of the device 100 apart from the first and second chambers 104, 108 may be covered. FIG. 2 shows an area A of the device 100 that may be essentially covered. One or more (e.g. all) functional units 102 of a device 100 may be covered, where the covering is applied to at least the first channel 106, second channel 110, and microchannel array 114.

FIG. 2 shows the operational part of the channels, which essentially corresponds to the width of the microchannel array L₁. The operational part of the first and second channels may therefore refer to the part of the channels that are in fluid connection via the microchannel array 114, and the length of the operational parts of the channels may be defined by the width L₁ of the microchannel array 114.

According to an embodiment the functional unit may be constructed such that the operational part may include the microchannel array 114, the first channel 106 in fluid connection with the microchannel array 114, and the second channel 110 in fluid contact with the microchannel array 114. The portion of the first channel 106 that is in fluid contact with the microchannel array 114 may be at least 75%, preferably at least 95%, even more preferably 100% of the total length of the first channel 106. The portion of the second channel 110 that is in fluid contact with the microchannel array 114 may be at least 50%, preferably at least 75%, even more preferably at least 95% of the total length of the second channel 110.

The width L₁ may be defined by the number of separate numbers of microchannels, their dimensions, and/or their spacing. The width of the microchannel array L₁ may be dependent on the accuracy of the automatic pipetting device used to load the chambers on either side of the microchannel array. The width of the microchannel array L₁ may be 30-9000 μm, preferably 3000-7000 μm, more preferably 4000-6000 μm.

According to an embodiment the width of the microchannel array L₁ may also be 30-2000 μm, preferably 50-100 μm. This may be advantageous when sample volumes are very small, but a high concentration ratio between the cell volume and the total volume is required by the assay.

FIG. 3 is an enlargement of a portion of the tilted side view of the embodiment of the microfluidic chip device in FIG. 2. Depicted are the first channel 106, second channel 110, and microchannel array 114.

The height of the second channel h₃ may be 10 μm-300 μm, preferably 170 μm-190 μm, such as 189 μm. The height of the first channel may be in the same range and may be equivalent to the height of the second channel. The height of the first and/or second channel may be optimized for better imaging (smaller focus depth) and to provide more nutrients for the cells compared to the situation in the prior art. If the device is made of only a single layer slide, for example PDMS, there is a trade-off between having a low chamber height for better imaging or a high chamber height for providing more nutrients. If additional cylindrical sample holder rings are attached to the chamber 104 and the chamber 108, it is possible to achieve a thinner, better imageable structure with larger sample holder parts. If a higher magnification is required, the thickness of the polymer part, i.e. the distance between the channel ceiling and the upper surface of the substrate, should be selected such that it will enable the closer proximity of the microscope lens of higher magnification in which the focus depth is much smaller.

The width of the first channel 106 may be 200-3000 μm, preferably 1190 μm. The first channel may be used for immune cells or the assay cell type that has higher volume and/or easier attainability. The second channel 110, may have a width of 100-1000 μm, preferably 490 μm. The second channel 110 may be used for cancer cells or assay cell type that has smaller volume and/or limited attainability.

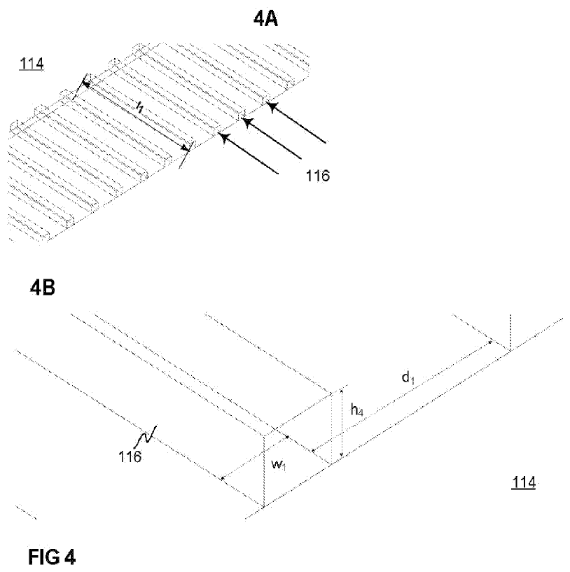

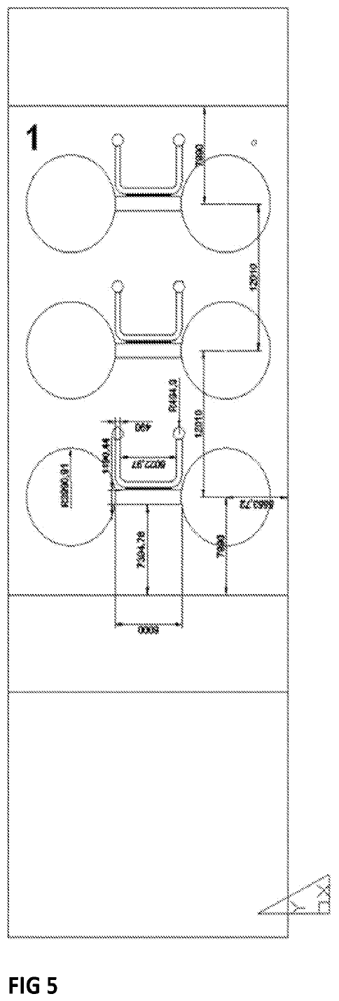

FIG. 4 shows enlargements of the tilted side view of a portion of one embodiment of the microfluidic chip device 100. Specifically, FIGS. 4A and 4B depict portions of a microchannel array 114 according to one embodiment of the invention.

The microchannel array 114 comprises a plurality of microchannels 116. Each microchannel 116 may have a height h⁴ of 5-20 μm, preferably 10-15 μm.

The microchannels 116 may be separated by a distance d₁ of 20-40 μm, preferably 27-37 μm, such as 32 μm. One intended function of the spacing or distance between microchannels is to separate cells from each other when entering the microchannel and leaving the microchannel on the other side. This may prevent any interaction or blocking the microchannel at the exit points by cells (e.g. cancer cells) by cells leaning over the neighboring microchannel.

The microchannels 116 may have a width w₁ of 10-15 μm, preferably 11-13 μm, e.g. 12 μm.

The length l₁ of each of the separate microchannels 116 in the microchannel array may vary. For the purpose of achieving different spatial gradient scenarios for chemical signaling and migration distance, several different lengths l₁ may be employed. The length l₁ of the microchannel 116 (or microchannel array 114) may be 10-2000 μm, preferably 10-1000 μm. For choosing the length l₁ of the microchannel (array) and the number of microchannels 116 the length of the first and/or the second channel on the two sides of the microchannel array may be taken into account (or at least the portion of the lengths of the first and second channels that are in physical contact with the microchannel array 114). The lengths of the first and/or second channels may be determined by the sample volume(s). The volume may be 1 μL-1000 μL on both sides. The cell content of the cell suspension volumes may be 0.1-149 μL, preferably 0.1-2.9 μL, more preferably 0.5-2 μL, even more preferably 1-2 μL The number microchannels 116 may be 3-250. The microchannels may be grouped into sub-groups of unevenly distributed microchannels.

According to an embodiment the optimal volume for the second channel, for example cancer cell, side, may be 1-5 μL containing the cell suspension. If the second channel is filled with tumor spheroids or organoids the channel volume may range between 5-25 μL. The first channel side, which typically contains the immune cell suspension or other type of cells that are in a larger volume, the optimal volume may be 20-1000 μL, typically 50-200 μL.

The microchannels 116 of the microchannel array 114 may be probed and filled with liquid/gel from both sides driven by capillary forces. Air pockets may remain in the microchannels. They typically disappear through dissolving into the media and/or being pushed out spontaneously through the nanopores of the polymer matrix of the device. This pushing is facilitated by the capillary forces pulling in the liquid from both sides into the microchannels. Another important requirement is that the liquids are not entering the microchannels while filling the channels. This feature is influenced by the surface properties of the polymer used facing the inner cavity of the microchannels Material choices and modification of surface properties may be used to avoid the formation of permanent air bubbles and to facilitate the removal of the air pockets trapped inside the device.

According to the embodiment in FIGS. 1-4, the second channel 110 is arranged in two 90 degree angles. This is not compulsory. The second channel 110 may be straight or have any angles providing comfortable spacing between the second chambers 108, that is providing enough space for convenient and safe pipetting.

FIG. 5 is a top view of one embodiment of the microfluidic chip device on a microscope slide. The surface area or radii of the chambers (first and second) may vary based on the size of the pipette tip radii and the microscope slide width.

The surface area of each of the first chambers 104 and the surface area of the each of the second chambers 108 may correspond to an essentially circular cross section with a radius between 0.5-10 mm. If the chambers 104, 108 were larger than that, fitting them on one standard microscope slide without affecting the stability of the architecture, also affecting ease of assembling of the device, would become challenging.

According to an embodiment the surface area or the radius of each of the second chambers 108 may be small enough, typically corresponding to an essentially circular cross section with a radius of 200-400 μm or matching the outer diameter of the radius of the loading device (e.g. a pipette), to provide sealing for the pipette tip when the cancer cell or other type of cell suspension is injected. The proper sealing is crucial because it prevents bubble formation when injecting.

According to an embodiment the surface area of each of the first chambers 104 may be larger than the surface area of each of the second chambers 108. Each of the first chambers 104 holds extra liquid both for the purpose of providing nutrient supply for the cells (typically immune cells) injected into the channel 106 prior to filling the reservoir and to mitigate the effect evaporation. Typical range for the surface area of the first chamber 104 may correspond to an essentially circular cross section with a radius of 3-5 mm.

The larger surface area of the pipetting area of the first chambers 104 may serve also as an open air container for media with cell nutrients for the immune cell suspension capable of providing better gas exchange between the liquid and atmosphere of the incubator.

The smaller pipetting area of the second chambers 108 may limit the evaporation of the gel sample injected in the narrower second channel and therefore preventing the shrinkage of the channel content. The inlet radii can be chosen depending of the physical properties of the loaded suspension and the cell types (if they require to be in gel matrix for achieving better viability, the number of cells and the gas exchange needs of the suspension.

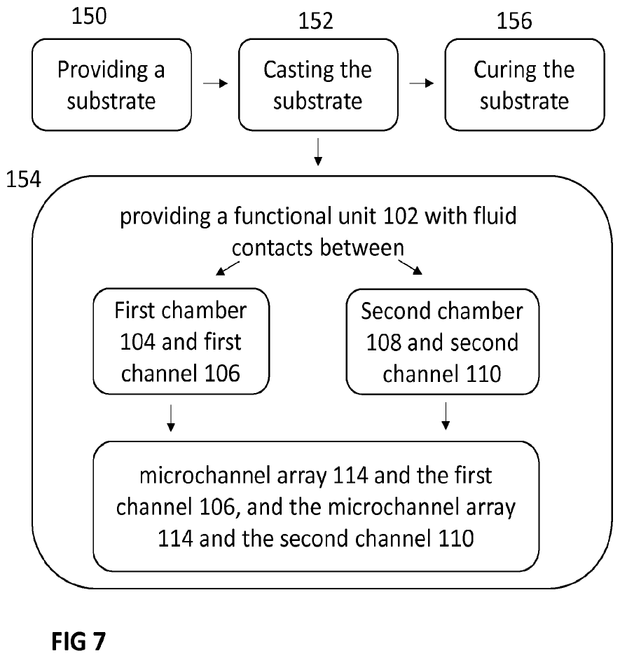

Similar dimensions of individual functional units 102 may be used when arranging the microfluidic device 100 on a microplate. A functional unit 102 may be independently provided for each of the wells of the microplate. Alternatively, a microplate sized flat surface, such as glass, may be used and each of the functional units 102 may be arranged on the flat surface according to the method described in FIG. 7 and the accompanying description in this application. If flat surface is used, the location of each of the functional units 102 may be arranged to coincide with the location of wells on a microplate with desired number of wells. For example, if the microfluidic device 100 is arranged to have 96 functional units 102, the locations of the functional units 102 may be arranged to match the well locations of a 96 or a 384 well microplate. This may allow microplate reader to have, e.g., 1, 2, or 4 reading locations from each of the functional units 102. Regardless of how the functional units 102 are assembled onto the microplate, the resulting solution enables repeated and automated analysis of all the functional units 102, each defined as a separate well or a corresponding well location.

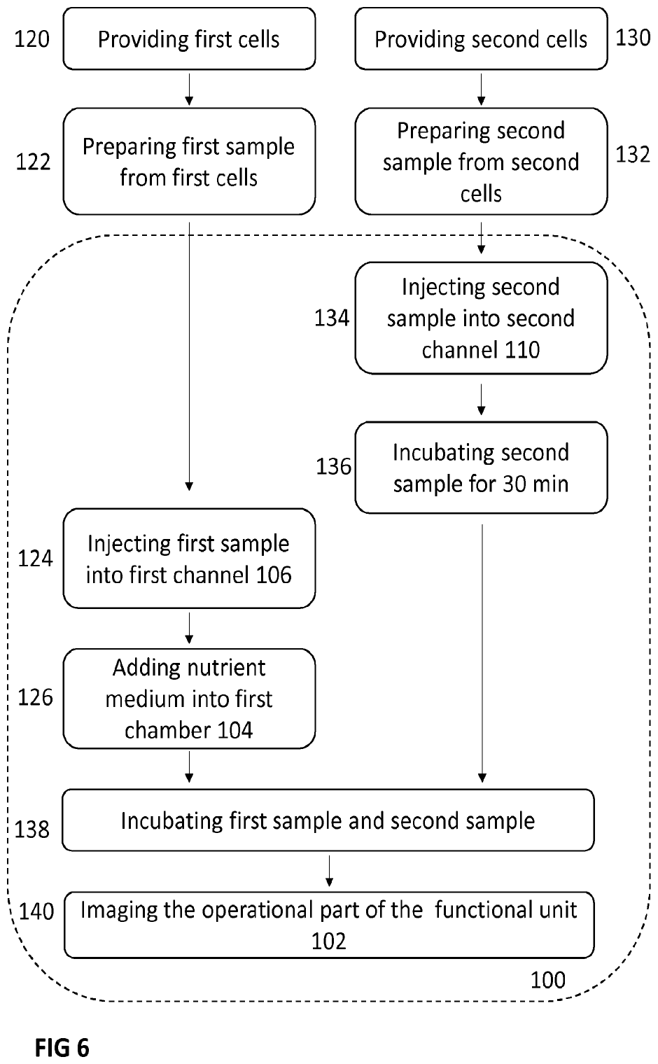

FIG. 6 is a flow chart of a method of culturing cells according to one embodiment of the invention. First cells are provided 120 for the cell culture e.g. from human peripheral blood mononuclear cells. The first cells are prepared 122 to form a first sample from the first cells. Second cells are provided 130 for the cell culture e.g. from cancer cells or organoids. The second cells are prepared 132 to form a second sample from the second cells. The second sample is injected 134 into the second channel 110 on the device 100. The device with the second sample is incubated 134 in a cell culture incubator, for example for 30 minutes. Thereafter, the first sample is injected 124 into the first channel 106 on the device 100. Nutrient medium is added 126 into the first chamber 104 to secure survival and osmolarity of the first cells of the first sample. After both of the channels 106 and 110 have received their samples, the device with the samples is incubated 138, for example for 12-72 hours. After the incubation, first cells from the first sample and second cells from the second sample have migrated into the microchannel array 114 of the operational part of the functional unit 102 and samples are imaged 140.

According to an exemplary embodiment, sample to be injected into second channel 110 may be prepared from tumour tissue samples, or corresponding samples, such as organoids. The sample to be studied may be obtained via biopsy or perioperatively or by other means. If the samples are tumour samples, they may be taken from the area adjacent to the centre of the tumour to assure the presence of the tissue cells of interest and cancer associated fibroblasts. Vital pieces of the sample may be isolated, preferably to 1-2 mm³, and then minced. The sample pieces may be minced into ice-cold Hanks' Balanced Salt solution (HBSS; supplied with 100 U/ml penicillin, 100 μg/ml streptomycin and 250 ng/ml fungizone). Then the sample pieces may be centrifuged for 5 min at 1000 rpm (200×g) at 4° C. Then the supernatant may be discarded and a fresh HBSS buffer may be added before repeating the centrifugation step. Then the tissue piece pellet may be suspended in a 5 ml HBSS buffer containing 1 mg/ml collagenase type I from Clostridium histolyticum and placed on a rocker platform at 37° C. for 2 h. The tube may then be centrifuged and the supernatant may be discarded and replaced with a fresh HBSS buffer before another round of centrifugation. The digested sample may be suspended in HBSS buffer, filtered using a 100-μm cell strainer and the flow-through single cells may be collected and centrifuged. The supernatant may be discarded and the cell pellet may be suspended in DMEM/F-12.

According to an exemplary embodiment the cell pellet from cancer cells or organoids may be stained e.g. with Celltrace Far Red and suspended in a solution that is liquid in room temperature in a media. This may be performed to form a hydrogel ECM after being injected into channel 110. The solution may be prepared from the following components with the following concentrations: 2.4 mg/ml Myogel, 0.5 mg/ml fibrinogen, 0.3 U/ml thrombin, and 33.3 μg/ml aprotinin; these reagents may be diluted in DMEM/F12 media with 10% patient's serum and immune checkpoint inhibitor drugs. For example, PD-1 inhibitor or fluorescent proteins and biosensors for super-resolution microscopy imaging or genetically encoded optogenetic tools for manipulation of biological processes with light may be added. Possible alternatives for Myogel include Lymphogel, ECM gel (Sigma), Cultrex® BME (Amsbio), Geltrex® (Gibco Life Technologies) and ECMatrix™ (Millipore), which products have the same disadvantage for human studies, because they are mouse tumor tissue homogenates that differ in composition from human TMEM. Then additional ingredients may be added in quantities resulting cell numbers and concentrations in the sample suitable for the given microchannel dimensions and volume which may be set to maximize the number of cells introduced into the channel cavity. The additive ingredients may prevent the formation of cell clusters. If organoids are used, instead of cancer cells, the organoids may be applied in concentrations which enable their introduction into the microchannel without forming aggregates.

According to an exemplary embodiment, sample to be injected into first channel 106 may be prepared from human peripheral blood mononuclear cells (MNCs), or corresponding samples. The sample may be isolated from a target tissue or in vitro cell culture. If human peripheral blood MNCs is used, they may be isolated from buffy coat of the cancer patient. The MNCs may be isolated via density gradient technique. The peripheral blood MNCs consists of immune cells of adaptive and innate types (T cells, B cells, NK cells, monocytes, and dendritic cells) individually or as a mixture of those in any combination. T cells may be separated and further processed according to CAR-T cell therapy protocols. Then they may be used alone or in combination with other immune cells for in vitro assessment of the efficacy assessment as a monotherapy or combination therapy. The process of incorporating the chimeric antigen receptor (CAR) encoding gene into the T cell, may be conducted according to any protocol defined as part of any CAR-T cell therapy. Serum from the cancer patients may be prepared by allowing it to clot at room temperature for 30 min and separating it via centrifugation at 2000 rpm for 10 min in a refrigerated centrifuge at 4 C. This serum may be added in 10% in volume when preparing the immune cell suspension. Immune stimulant and immune checkpoint inhibitor drugs or fluorescent proteins and biosensors for super-resolution microscopy imaging or genetically encoded optogenetic tools for manipulation of biological processes with light may be added. Other ingredients may be added in quantities resulting cell numbers and concentrations in the sample suitable for the given microchannel dimensions and volume which is set to maximize the number of cells introduced into the microchannel cavity. The additive ingredients may prevent the formation of cell clusters, aggregates.

According to an exemplary embodiment, the sample with cancer cells suspension may be loaded into the second channel 110. Then the chip may be placed into an incubator for 30 minutes resulting the ECM hydrogel to be formed. Then the sample with immune cell suspension may be loaded into first channel 106. Following the direct injection of the cell suspension to the first channel 106 additional buffer may be loaded to the first chamber 104 to provide nutrients and prevent the drying out of the first channel 106 during incubation. According to an embodiment, the device 100 may be placed into a cell culture incubator after both channels 110 and 106 have been loaded. In the 12 hours following the placement of the device 100 into the cell culture incubator the microchannel array 114 between the parallel sections of first channel 106 and second channel 110 forming the operational part of the functional unit 102 may be primed and the air bubbles trapped in the microchannels 116 may be eliminated. After the incubation the device 100 may be imaged by fluorescence microscope several times over the next 36 hours. During this time an immune response towards the cancer cells is formed and the immune cells are migrating towards the cancer cells via the microchannels 116. When reaching the cancer cells, due to the blocked immune checkpoints, the cancer cells may be neutralized. While imaging the device 100 the number of immune cells passing through the microchannel array 114 may be counted and may serve as a direct indication of the immune response. The cancer cells neutralized by the immune cells may be observable via the change of colour in their fluorescence. The number of those cancer cells attacked and killed by immune cells may be a direct indication of the efficacy of treatment with the drug therapy or combinatory drug therapy. It is obvious to a person skilled in the art that the procedure described above may be repeated in parallel in several functional units thereby enabling duplicate and reference sampling or sampling of different dosages, cells, or drugs at the same time.

According to an embodiment several drugs and/or drug candidates, or combinations of drugs and/or drug candidates may be used. They may include, but are not limited to, the drugs and drug candidates listed below and in the following tables 1-4.

TABLE 1
Exemplary drugs and drug candidates with inhibitory
function on immune checkpoint targets

| Target | Drug |
|---|---|
| LAG-3 (CD223) | LAG525 (IMP701), REGN3767 (R3767), BI |
| | 754,091, Tebotelimab (MGD013), Eftilagimod |
| | alpha (IMP321) and FS118. |
| TIM-3 | MBG453, Sym023, TSR-022 |
| B7-H3 and B7-H4 | MGC018, FPA150 |
| A2aR | EOS100850, AB928 |
| CD73 | CPI-006 |
| NKG2A | Monalizumab |
| PVRIG/PVRL2 | COM701 |

TABLE 2
Drugs and drug candidates with inhibitory
functions beyond immune checkpoint targets

| Target | Drug |
|---|---|
| CEACAM1 | CM24 |
| CEACAM 5/6 | NEO-201 |
| FAK | Defactinib |
| CCL2/CCR2 | PF-04136309 |
| LIF | MSC-1 |
| CD47/SIRP | Hu5F9-G4 (5F9), ALX148, TTI-662, RRx-001 |
| CSF-1 (M-CSF)/CSF1R | Lacnotuzumab (MCS110), LY3022855, |
| | SNDX-6352, Emactuzumab (RG7155), |
| | Pexidartinib (PLX3397) |
| IL-1 and IL-1R3 (IL- | CAN04, Canakinumab (ACZ885) |
| 1RAP) | |
| IL-8 | BMS-986253 |
| SEMA4D | Pepinemab (VX15/2503) |
| Ang-2 | Trebananib |
| CLEVER-1 | FP-1305 |
| Axl | Enapotamab vedotin (EnaV) |
| | Phosphatidylserine |

TABLE 3
Existing immune checkpoint inhibitor drugs

| Target | Drug |
|---|---|
| CTLA-4 | Ipilimumab |
| PD-1 | Cemiplimab, Nivolumab, Pembrolizumab |
| PD-L1 | Atezolizumab, Avelumab, Durvalumab |

TABLE 4
New immune checkpoint inhibitor molecules
and other inhibitory targets

| Target | Agent |
|---|---|
| LAG-3 (CD223) | LAG525 (IMP701), REGN3767 (R3767), BI |
| | 754,091, tebotelimab (MGD013), eftilagimod |
| | alpha (IMP321), FS118 |
| TIM-3 | MBG453, Sym023, TSR-022 |
| B7-H3, B7-H4 | MGC018, FPA150 |
| A2aR | EOS100850, AB928 |
| CD73 | CPI-006 |
| NKG2A | Monalizumab |
| PVRIG/PVRL2 | COM701 |
| CEACAM1 | CM24 |
| CEACAM 5/6 | NEO-201 |
| FAK | Defactinib |
| CCL2/CCR2 | PF-04136309 |
| LIF | MSC-1 |
| CD47/SIRPα | Hu5F9-G4 (5F9), ALX148, TTI-662, RRx-001 |
| CSF-1 (M-CSF)/CSF-1R | Lacnotuzumab (MCS110), LY3022855, |
| | SNDX-6352, emactuzumab (RG7155), |
| | pexidartinib (PLX3397) |
| IL-1 and IL-1R3 (IL- | CAN04, Canakinumab (ACZ885) |
| 1RAP) | |
| IL-8 | BMS-986253 |
| SEMA4D | Pepinemab (VX15/2503) |
| Ang-2 | Trebananib |
| CLEVER-1 | FP-1305 |
| Axl | Enapotamab vedotin (EnaV) |
| Phosphatidylserine | Bavituximab |

In addition to drugs and drug candidates listed in tables 1-4, Chinese domestically developed immune checkpoint inhibitor drugs and drug candidates Tuoyi (toripalimab), Tyvyt (Sintilimab), Tislelizumab, Camrelizumab, AK105, CS1001, CS1003, zimberelimab, HLX-10, KN046, SHR-1316 may be used. Also other Humanized lgG4 anti PD-1 monoclonal antibody drugs and drug candidates, such as Spartalizumab (PDR001), Dostarlimab, TSR042, MGA012, Sasanlimab (PF-06801591), Budigalimab (ABBV-181), BI754091 may be used. In addition, subcutaneously administered PD-L1 nanobody Envafolimab (KN035), fully human lgG1 PD-L1 monoclonal antibody drug or drug candidate Cosibelimab (CK-301), bifunctional fusion protein, TGFβRII extracellular domains fused to human lgG1 PD-L1 monoclonal antibody drug or drug candidate Bintrafusp alfa (M7824), and/or small molecule inhibitor of VISTA, PD-L1, and PD-L2 CA-170 may be used

FIG. 7 is a flow chart of a method of manufacturing a device according to one embodiment of the invention. A substrate is provided 150 to manufacture the device. The substrate is casted 152 to form the necessary fluid connectivity within the device. The fluid connectivity is provided 154 between first chamber 104 and first channel 106, between second chamber 108 and second channel 110, between microchannel array 114 and the first channel 106, and between the microchannel array 114 and the second channel 110. Finally, the substrate is cured 156 to have a functional device 100.

The method for preparing the microfluidic device 100 may be based on standard soft lithography as described in the prior art. The substrate thickness may be 30 μm-3.5 mm. Locally thinner substrate thickness may result in increased gas permeability as there is a shorter vertical distance for the gas bubbles to escape from the microchannels.

Because the microchannel array 114 is loaded from both the first channel 106 side and the second channel 110 side, the air bubbles are forced to remain within the microchannel array 114. Therefore, the gas bubbles that are trapped into the microchannels 116 before and during the loading of the samples remain within the microchannel array 114. Increased gas permeability may allow the gas bubbles to escape faster thus enabling also a quicker priming of the microchannel array 114.

In order to provide adequate level of gas permeability for the cell culturing and to facilitate the elimination of air bubbles from microchannels during priming of the device 100, PDMS or other materials with equivalent gas permeability are preferred to be used for the cell culturing and cell migration functional parts of the device 100. The PDMS structural parts can be put into their intended shape by casting over a mold and thermo curing the material following its mixture with a crosslinking agent. Photocurable PDMS can be also used and photocuring can be applied after casting or as part of an additive manufacturing (3D printing) process. In addition Roll to Roll nanoimprinting can also be used for both processes involving the thermo or the photo curing processes or the combination of those.

Besides PDMS other thermoplastic materials can be used alone or in combination with each other to form both the structural and the functional parts of the device 100, including polystyrene, poly methyl metacrilate (PMMA), Poly(ethylene glycol) diacrylate (PEGDA), Cyclic Olefin Copolymer (COC), and Cyclic Olefin Polymer (COP) or any other thermoplastic or any combination of these materials. These materials can be used to put into shape of the intended channel and supporting structures of the main chip architecture via microinjection molding or hot embossing. Roll to roll embossing or imprinting is another way to mass manufacture.

Molds for the casting or molding can be prepared by additive manufacturing, CNC micro-milling, electroplating, or lithographic methods which may involve various ways of etching or adding materials to the molding tool forming the microchannel structure to be replicated.

Many variations of the present application will suggest themselves to those skilled in the art in light of the description in previous. Such obvious variations are within the full intended scope of the appended claims.

1. A microfluidic device, comprising:
a gas permeable substrate
one or more functional units each functional unit comprising
two first chambers arranged to be in fluid contact by a first channel,
two second chambers arranged to be in fluid contact by a second channel,
a microchannel array arranged to connect the first channel and the second channel, and
wherein the microchannel array, the portion of the first channel in fluid connection with the microchannel array, and the portion of the second channel in fluid connection with the microchannel array form an operational part of each functional unit.

2. Microfluidic device according to claim 1, wherein each functional unit is a separated unit, not in fluid contact with any of the other functional units, and preferably arranged to be loaded independently.

3. Microfluidic device according to claim 1, wherein a width of the microchannel array (L₁) is in the range of 30-9000 μm, preferably 3000-7000 μm, more preferably 4000-6000 μm.

4. Microfluidic device according claim 1, wherein a width of the microchannel array (L₁) is in the range of 30-2000 μm, preferably 50-100 μm

5. Microfluidic device according claim 1, wherein the device is arranged on a slide for microscopy.

6. Microfluidic device according to claim 1, wherein the device is arranged on a microplate.

7. Microfluidic device according claim 1, wherein at least a portion of the device, optionally essentially all or most of the device apart from the first and second chambers, is transparently covered.

8. Microfluidic device according claim 1, wherein each of the first chambers have height of 100 μm-3 cm, and an essentially circular cross section with a radius of 0.5-10 mm, preferably 3-5 mm or an area spanning over 0.7-314 mm², preferably 28-78.5 mm² of any other 2D shape.

9. Microfluidic device according claim 1, wherein each of the second chambers have height of 100 μm-3 cm, and a surface area corresponding to an essentially circular cross section with a radius of 200-400 μm or a radius that corresponds to the outer diameter of the radius of the loading device that is used to load each of the second chambers.

10. Microfluidic device according to claim 1, wherein the first channel has height of 10 μm-300 μm, preferably 170 μm-190 μm, such as 189 μm, the first channel (106) has a width 200-3000 μm, preferably 1190 μm, the second channel has height of 10 μm-300 μm, preferably 170 μm-190 μm, such as 189 μm, the second channel has a width of 100-1000 μm, preferably 490 μm, the length of the first channel is at least the length of the microchannel array, and the length of the second channel is at least the length of the microchannel array.

11. Microfluidic device according to claim 1, wherein each of the microchannels in the microchannel array have
height (h4) of 5-20 μm, preferably 10-15 μm,
width (w1) of 10-15 μm, preferably 11-13 μm,
length (l1) of 10-2000 μm, preferably 10-1000 μm, and
20-40 μm, preferably 27-37 μm distance (d1) between microchannels.

12. Microfluidic device according to claim 1, wherein the device comprises 1-384 functional units.

13. Microfluidic device according to claim 1, wherein the device comprises PDMS.

14. A method for culturing cells, the method comprising providing a device according to claim 1, providing at least one first chamber with at least immune cells, providing at least one second chamber with at least cancer cells, incubating the device and counting the immune cells and/or cancer cells while observing the migration of the immune cells to the cancer cells.

15. The method of claim 14, comprising providing the at least one first chamber with at least immune cells and/or providing the at least one second chamber with at least cancer cells by one or more automatic pipettes.

16. A method of manufacturing a microfluidic chip comprising
providing a substrate
limiting the thickness of the substrate to 30 μm-3.5 mmworking the substrate to define one or more functional units each functional unit comprising
two first chambers arranged to be in fluid contact by a first channel,
two second chambers arranged to be in fluid contact by a second channel,
a microchannel array arranged to connect the first channel and the second channel,
an operational part arranged to connect the portion of the first channel in fluid connection with the microchannel array, and the portion of the second channel in fluid connection with the microchannel array,
curing the substrate
